(19) United States
(12) Patent Application Publication
(10) Pub. No.: US 2021/0386056 A1
(43) Pub. Date: Dec. 16, 2021

(54) TISSUE SAMPLE CORING SYSTEM

(71) Applicant: University of Washington, Seattle, WA (US)

(72) Inventors: Karol Bomsztyk, Seattle, WA (US); Daniel Mar, Seattle, WA (US); Stephen Scheuerman, Seattle, WA (US); Greg Darlington, Seattle, WA (US)

(21) Appl. No.: 17/348,504

(22) Filed: Jun. 15, 2021

Related U.S. Application Data
(60) Provisional application No. 63/039,679, filed on Jun. 16, 2020.

(51) Int. Cl. A01N 1/02; A61B 10/00

(52) U.S. Cl. CPC A01N 1/0263; A01N 1/0257; A61B 10/0096

(57) ABSTRACT

The present technology generally relates to a cryogenic tissue sample storage system. Select embodiments of a tissue sample storage system include a tray having a first well configured to receive a first tissue sample therein a second well configured to receive a second tissue sample therein. The tray can include a first well identification feature associated with the first well and a second well identification feature associated with the second well. The storage system can further include a lid hingedly associated with the tray and movable between (a) a first position in which the first and second wells are occluded by the lid, and (b) a second position in which the first and second wells are externally accessible. The first well identification feature can be unique from the second well identification feature.

### CROSS-REFERENCE TO RELATED APPLICATION(S)

The present application claims priority to U.S. Provisional Patent Application No.: 63/039,679, titled “PLATFORM FOR FREEZING AND STORING TISSUE,” filed Jun. 16, 2020, the disclosure of which is hereby incorporated by reference in its entirety.

### STATEMENT OF FEDERALLY SPONSORED RESEARCH AND DEVELOPMENT

This present technology was made with government support under R33 CA191135, R01 DK103849, R21 GM111439, R44 GM122097, R42 HG010855, and U01 CA246503, awarded by National Institutes of Health. The government has certain rights in the invention.

### TECHNICAL FIELD

The present technology generally relates to systems and methods for storing cryogenic tissue samples in a storage container and identifying the samples.

### BACKGROUND

Histological and molecular intra-tissue heterogeneity is present in virtually every disease, including cancer and organ injury. This heterogeneity may account for many therapeutic failures (particularly in cancer), and is shifting the paradigm that multiple, as opposed to single, biopsies are needed to optimize personalized medical care. There are powerful high throughput pre-analytical sample preparation and analytical platforms to study intracellular processes and their alterations in tissues, including molecular intratissue heterogeneity in cancer and organ injury. Freezing or paraffin embedding of formalin fixed tissues (FFPE) is a common way to preserve and store samples for analysis (e.g. surgical specimens for pathologist evaluation).

Advances in high throughput (HT) sample preparation and analytical technologies are providing opportunities to study intra-tissue heterogeneity, leading to discoveries of disease biomarkers. Tissue biopsies are among the most common medical procedures used to establish diagnosis. Historically, tissue biopsies were primarily used for histology. More recently, with increasing understanding of molecular basis of disease, tissue samples are being used in personalized medicine where treatments are based on discoveries of molecular biomarkers.

Space shortages in deep freezers (e.g., −80° C. or less) can require discarding of older samples to make room for new samples. Cryogenic tissues are typically stored in tubes (often 1.5 ml), but typically only a small space is occupied on the bottom tip of the tube, leading to inefficient freezer storage. The frozen tissue samples freeze attached to the bottom of the tube, and removal often requires a degree of thawing. Repeated thawing and freezing can cause sample degradation.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is an exploded perspective view of a tissue sample storage container grid assembly configured in accordance with an embodiment of the present technology.

FIG. 2 is an enlarged perspective view of the tissue sample storage container of FIG. 1, showing a compartment lid open.

FIG. 3 is a perspective view of a tissue sample storage container grid configured in accordance with an embodiment of the present technology.

FIG. 4 is a front view of the tissue sample storage container grid of FIG. 3.

FIG. 5 is a front view of a tray portion of the tissue sample storage container grid of FIG. 3, showing an identifying corner.

### DETAILED DESCRIPTION

### A. Overview

The present technology is directed to a cryogenic tissue sample storage system having a tissue sample storage container grid. Storage systems of the present technology increase the density of tissue samples per volume to allow a greater number of samples to be stored in freezers. The relatively shallow storage wells increase accessibility of the samples and avoid the degradation of a thaw and refreeze cycle of the cryogenic sample. Under certain testing protocols, it is necessary to maintain the cores in a frozen state to preserve the normal and/or diseased cell conditions, preventing the cells from changing state during preparation, transportation, and/or storage of the samples. Tissue sample storage containers configured in accordance with the present technology maintain the cores in a frozen state and provide catalog information for identification of the cores in each well.

### B. Selected Embodiments of a Cryogenic Tissue Sample Storage Container

Frozen tissue sample blocks from which cores are extracted by a coring tool (e.g., the coring tools described in International Patent Application No. PCT/US2019/057835, filed Oct. 24, 2019, the entirety of which is incorporated by reference herein), may be contained within a grid of cryogenically temperature-controlled sample wells. Storage container grids configured in accordance with the present technology may be adapted to store and organize tissue samples in a space-efficient manner for coring and testing. The frozen tissue samples can be kept in a frozen state by any suitable heat removal method, such as placing the storage grid in an enclosure (e.g., a freezer) or a chilled component, and/or contacting the storage grid with a cold substance (e.g., solid carbon dioxide, “dry ice,” etc.). In some embodiments, the storage grid may have active heat removal to keep the tissue samples at a specified temperature for long-term storage.

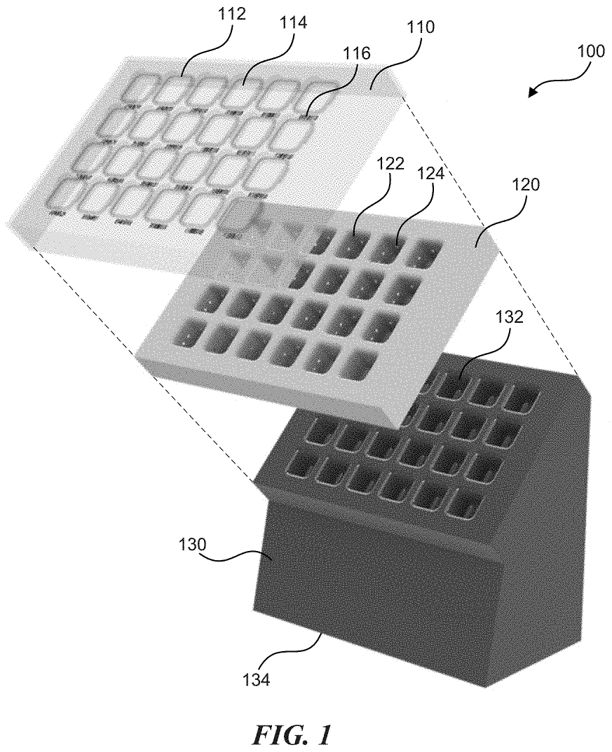

FIG. 1 is an exploded perspective view of a tissue sample storage container grid assembly 100 (“grid assembly 100”) configured in accordance with an embodiment of the present technology. The grid assembly 100 includes a lid 110, a tray 120, and a base 130. The components of the grid assembly 100 are separable, such that one or more components may be used without the full assembly, e.g., using the lid 110 and the tray 120 together and placing the subassembly of the lid 110 and the tray 120 in a freezer without the base 130.

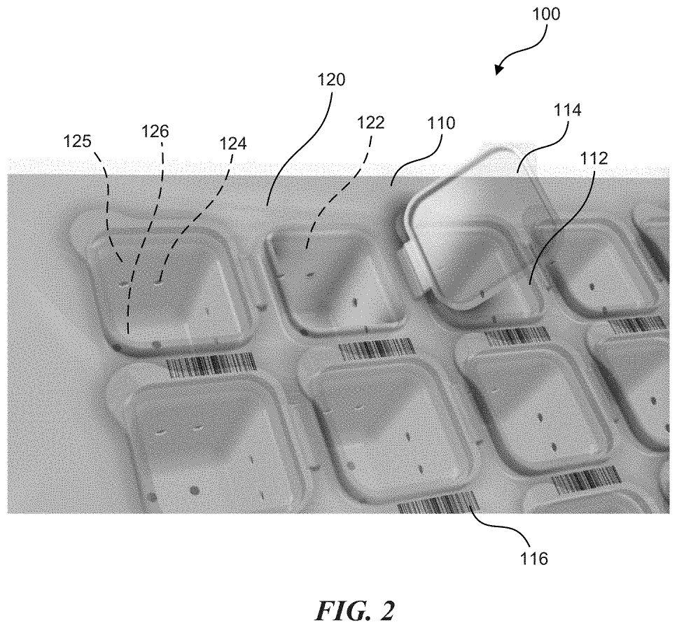

The lid 110 has an array of individual openings 112 generally arranged in a grid along the surface of the lid 110. Each of the openings 112 can include a cover or semi- to fully transparent door 114 configured to occlude the corresponding opening 112 when the opening is not in use, after the tissue samples have been placed within the grid assembly 100, during storage, etc. The door 114 can be hingedly coupled to the lid 110 (as shown in FIG. 2) or can be detachable from the lid 110. The lid 110 can further include an identification feature (e.g., a barcode 116) near each of the openings 112 configured to identify the tissue sample stored within a particular position of the grid assembly 100.

The tray 120 includes an array of wells 122 generally corresponding to the position of the openings 112 of the lid 110. The wells 122 are sized and configured to enclose tissue samples together with the doors 114. Although each well 122 is shown in the Figures having a rounded-rectangular shape, the wells can have any suitable shape, such as circular, elliptical, triangular, geometric, etc. Each well 122 is defined by walls 125 extending from a perimeter aligned with the openings 112, and a bottom portion 126. The wall 125 and/or the bottom portion 126 of the wells 122 can include one or more perforations 124 to increase the heat transfer from the tissue samples inside of the wells 122 to the base 130, e.g., while extracting heat during freezing, or to another heat transfer component. The tray 120 can include one or more mechanical coupling features (not shown) that correspond to features on the lid 110 and provide a removable coupling of the lid 110 on the tray 120. In this regard the subassembly of the lid 110 and the tray 120 can be removed from the base 130 without separating the lid 110 and the tray 120, retaining the tissue samples within the wells 122. The tray 120 can be formed from plastic sheets (e.g., polystyrene, polycarbonate, etc.) and molded into skirted rectangular trays that contain the wells 122 for storing the tissue samples. The lid 110 and tray 120 can be made from any suitable material, such as a plastic (e.g., polystyrene, polypropylene, etc.), metal, etc.

The base 130 includes an array of pockets 132 generally corresponding to the position of the openings 112 of the lid 110 and the wells 122 of the tray 120. The pockets 132 are sized and configured to receive the wells 122 therein, and may contact one or more of the outer surfaces of the wells 122 (e.g., the walls 125 and/or the bottom portion 126) to increase heat transfer. As shown in FIG. 1, the base 130 can include a lower surface 134 configured to provide stability when the base 130 is in use, e.g., when the grid assembly 100 is placed on a counter, table top, or the bottom of a container having dry ice for freezing the grid assembly 100. In this regard, the lower surface 134 and the surface having the pockets 132 may be disposed at an angle from each other to improve ergonomics for the user during extraction of the cores from the tissue samples, e.g., by the coring tool of International Patent Application No. PCT/US2019/057835 that was previously incorporated by reference, or another suitable instrument. In some embodiments, the base 130 is formed from a material (e.g., metal, stone, etc.) with material density and heat transfer properties suitable for maintaining the tissue samples in the wells 122 at a desired temperature. In a similar manner as with the lid 110 and tray 120 described above, the base 130 can include one or more mechanical coupling features (not shown) that correspond to features on the lid 110 and/or tray 120 to provide a secure connection of the lid 110 and/or tray 120 on the base 130. The base 130 can have an aperture (not shown) on a side for insertion of a thermocouple to allow monitoring of the temperature of the base 130 to ensure the tissue samples in the tray 120 remain in a frozen state (e.g., −70° C. or less).

The grid assembly 100 can be placed within an additional enclosure, such as a freezer or cooler, to improve heat transfer and maintain the tissue samples at a desired temperature and/or for longer term storage. During placement of the fresh tissue samples within the grid assembly 100, the base 130 can be pre-chilled separately from the tray 120 and the lid 110. After chilling of the base 130, the fresh tissues are prepared and are placed in the wells 122. Optimal cutting temperature (OCT), Cryo-Gel, or other cryogenic embedding matrix may be added to the wells 122 to increase the heat transfer to the fresh tissue samples such that the OCT, Cryo-Gel, or other cryogenic embedding matrix solidifies around the samples for immobilization. In this regard, the embedding matrix may additionally fill the perforations 124 to create a mechanical lock within the well 122 for improved immobilization of the tissue samples during core extraction with, e.g., the coring tool of International Patent Application No. PCT/US2019/057835 as previously incorporated herein, or another suitable instrument. In other embodiments, the grid assembly 100 can be used to freeze the tissue samples at a slower rate than conventional methods (e.g., flash freezing), which can avoid damage to the tissue samples. If the lid 110 is removed for placement of the tissue samples and the OCT embedding matrix in the wells 122, the lid 110 can be replaced over the tray 120, and then the subassembly of the lid 110 and the tray 120 can be removed from the base 130 for storage in a freezer. In other embodiments, each individual door 114 can be opened during placement of the tissue samples and the cryogenic embedding matrix, and then closed for storage. In some embodiments, the cores are stored at or below −80° C. in a freezer.

To access the tissue samples for core extraction and testing, e.g., following storage, the subassembly of the lid 110 and the tray 120 may be placed on a pre-chilled base 130 to maintain the freezing state of the tissue samples while the cores are extracted from the tissue samples. During extraction, the doors 114 can be opened individually to extract the cores, e.g., the coring tool of International Patent Application No. PCT/US2019/057835 as previously incorporated herein, or another suitable instrument, without exposing the tissue samples in other wells 122 to the surrounding environment. In some embodiments, the barcodes 116 provide identification for the tissue sample contained in the wells 122. After the cores are extracted from the tissue samples, the subassembly of the lid 110 and the tray 120 can be removed from the base 130 are placed back in the freezer for further testing.

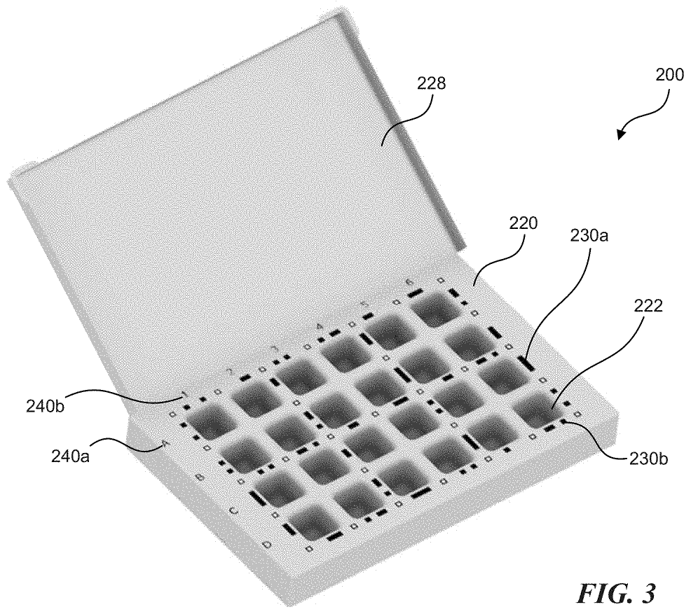

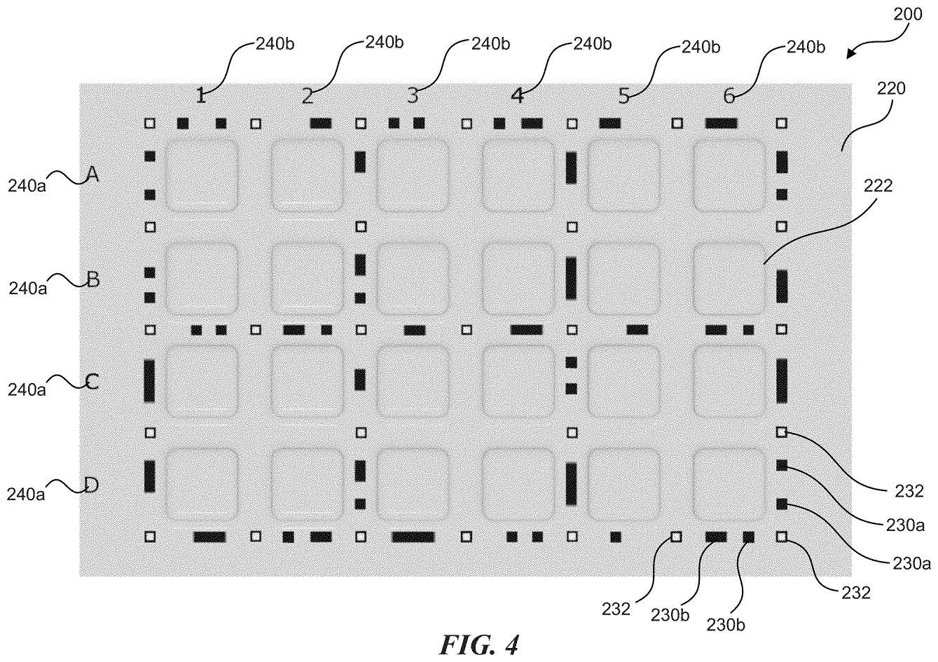

FIG. 3 is a perspective view of a tissue sample storage container grid 200 (“grid 200”) configured in accordance with an embodiment of the present technology. The grid 200 has similarities to components of the grid assembly 100, except the grid 200 generally combines the lid 110 and the tray 120 into a single component. The grid 200 includes a tray portion 220 and a lid portion 228 and can be configured to be used with a suitable base (e.g., the base 130 of the grid assembly 100). The tray portion 220 has a grid of wells 222 for retaining tissue samples therein. Although not shown in the illustrated embodiments of FIGS. 3 and 4, the wells 222 can further include perforations similar to the perforations 124 described above. The lid portion 228 can be hingedly associated with the tray portion 220 such that the lid portion 228 can be selectively closed to enclose the tissue samples within the wells 222. In other embodiments, the lid portion 228 may have individual doors corresponding the position of each of the wells 222, e.g., similar to the doors 114 described above with respect to the grid assembly 100. The grid 200 can include an identification feature on the lid portion 228 and/or the base portion (not shown), e.g., a barcode, matrix code (e.g., a QR code), etc. to aid in identification of the tissue samples within the grid 200.

FIG. 4 is a front view of the grid 200 and shows various human-readable and computer-readable well identification features, similar to the barcodes 116 of the lid 110 described above with respect to the grid assembly 100. In one example, a surface of the tray portion 220 can include human-readable well identification features, including well row markers 240a and well column markers 240b that can be used to identify the well 222, e.g., to determine which well 222 contains a certain tissue sample. For example, the well 222 marked “A5” may have a different tissue sample type than the well 222 marked “C2,” etc. The surface of the tray portion 220 can include computer-readable well identification features in addition to, or as an alternate to the human-readable well identification features. The computer-readable well identification features can be input to the computer by any suitable imaging device or scanner, e.g., a camera, a laser scanner, etc., and can be mounted to the coring tool of International Patent Application No. PCT/US2019/057835, as previously incorporated herein. The computer-readable well identification features can include a barcode-type system having vertical indicia 230a, horizontal indicia 230b, and separating/positioning indicia 232. The vertical, horizontal, and separating/positioning indicia 230a, 230b, 232 can be configured to allow a computer to identify the position of each well 222, and can be further configured for computer identification when a layer of frost has covered the grid 200 after freezing.

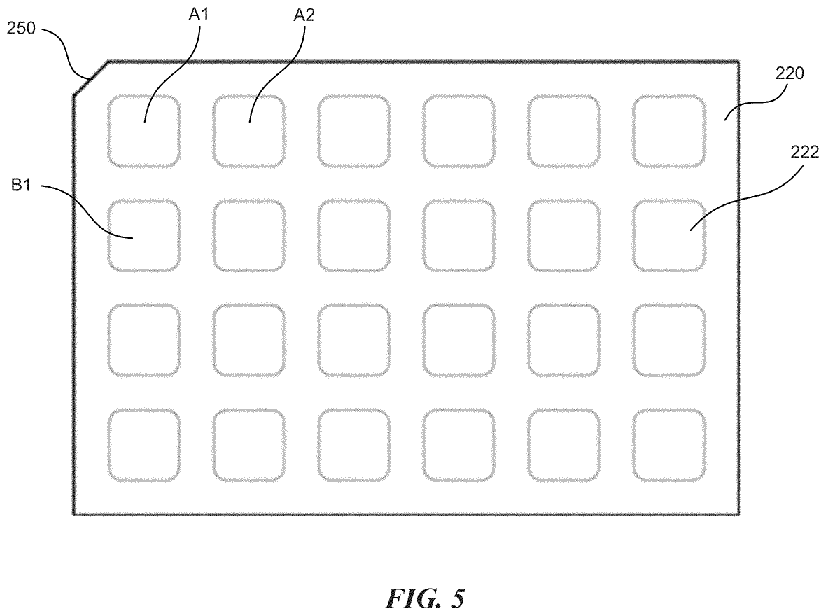

In other embodiments, the wells 222 can be identified using any suitable well identification features, such as cuts (see FIG. 5), markings (not shown), a series of colored portions (not shown), etc. suitable for identification by a computer (e.g., by a camera, scanner, etc.), where the camera and/or scanner unit can be coupled to a component of the grid 200 and/or positioned above the tray portion 220 to identify the wells 222. In other embodiments, any of the suitable well identification features (e.g., the well row markers 240a, the well column markers 240b, the vertical indicia 230a, the horizontal indicia 230b, the separating/positioning indicia 232, markings, colored portions, borders, etc.) can be positioned on a fixture (e.g., the base 130 or another suitable fixture) configured to carry the tray portion 220, such that a camera and/or scanner unit can interpret the well identifying features through the tray portion 220, e.g., by a transparent area of the tray portion 220 aligned with the well identifying features.

FIG. 5 is a front view of the tray portion 220 and shows an identifying corner 250 having a different configuration than the other three corners. The identifying corner 250 can allow the camera/scanner to determine the orientation of the tray portion 220 for identification of the position of a first well A1 nearest the identifying corner 250, and then relative identification of the other wells (e.g., A2, B1, etc.) in relation to the first well A1. The identifying corner 250 can be configured to interface with a corresponding feature in a fixture (e.g., a complementary feature in the base 130 or another suitable fixture) to orient the tray portion 220 relative to the fixture. A camera associated with the fixture would then be able to determine the position of the wells 222 of the tray portion 220 by the orientation of the tray portion 220 based on the identifying corner 250. In these embodiments, a further identifying feature (e.g., markings, colored portions, etc.) may not be required.

In embodiments with markings, the marking can be a dark border (not shown) around a first well 222 (e.g., the upper left well) which would give the camera/scanner an orientation of the tray portion 220 for relative identification of the other wells 222. In further embodiments, each of the wells could include a border to assist in identification of the well positions by the camera/scanner. In embodiments with colored portions, the color coding can include a partial or full colored border around the opening of the well 222, colored paneling within the well 222, a colored area on the lid portion 228 corresponding to the position of the well 222, or any combination thereof. In further embodiments, the OCT embedding matrix or other freezing media within the well 222 can include dyes/pigments used to identify the position of each well 222, and may be included with the grid 200 during manufacturing, such that a user would add a tissue sample to a pre-colored freezing media and then freeze the grid 200 with the identifying freezing media and tissue samples in each of the wells 222. For example, the well 222 corresponding to human-readable position “B4” can have a specific color hue to allow identification of the well 222 position by the computer. In any of the well identification embodiments described above, the well position data can be combined by the computer with image data from a tool, (e.g., the coring tool of International Patent Application No. PCT/US2019/057835, as previously incorporated herein), to determine which well 222 the tool is sampling.

### C. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Moreover, the various embodiments described herein may also be combined to provide further embodiments. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment.

For ease of reference, identical reference numbers are used to identify similar or analogous components or features throughout this disclosure, but the use of the same reference number does not imply that the features should be construed to be identical. Indeed, in many examples described herein, identically numbered features have a plurality of embodiments that are distinct in structure and/or function from each other. Furthermore, the same shading may be used to indicate materials in cross section that can be compositionally similar, but the use of the same shading does not imply that the materials should be construed to be identical unless specifically noted herein.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms may also include the plural or singular term, respectively. As used herein, with respect to measurements, “about” means +/−5%. Additionally, the term “comprising” is used throughout to mean including at least the recited feature(s) such that any greater number of the same feature and/or additional types of other features are not precluded. Directional terms, such as “upper,” “lower,” “front,” “back,” “vertical,” and “horizontal,” may be used herein to express and clarify the relationship between various elements. It should be understood that such terms do not denote absolute orientation. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

We claim:

1. A cryogenic storage container, comprising:
a tray, having
a first opening and a second opening;
a first wall projecting from a perimeter of the first opening, and a first bottom portion coupled to the first wall opposite the first opening, wherein the first wall and first bottom portion together define a first well configured to receive a first tissue sample therein; and
a second wall projecting from a perimeter of the second opening, and a second bottom portion coupled to the second wall opposite the second opening, wherein the second wall and second bottom portion together define a second well configured to receive a second tissue sample therein; and
a lid removably couplable to the tray and having a first door aligned with the first opening and a second door aligned with the second opening,
wherein the first and second doors are movable between (a) a first position in which the first and second openings, respectively, are occluded, and (b) a second position in which the first and second wells are accessible through the first and second openings.

2. The cryogenic storage container of claim 1, further comprising:
a first well identification feature associated with the first well; and
a second well identification feature associated with the second well,
wherein the first well identification feature is unique from the second well identification feature.

3. The cryogenic storage container of claim 2, wherein the first and second well identification features are configured to provide information related to one or more of content of the first and second wells, a position of the first and second wells relative to the tray, or a position of the first well relative to the second well.

4. The cryogenic storage container of claim 2, wherein the first and second well identification features comprise one or more of an indicia, a barcode, a matrix code, a cut, a marking, a color code, a letter, a number, or a symbol.

5. The cryogenic storage container of claim 2, wherein:
the first well identification feature comprises a first color at least partially surrounding the perimeter of the first opening,
the second well identification feature comprises a second color at least partially surrounding the perimeter of the second opening, and
the first color is a different hue than the second color.

6. The cryogenic storage container of claim 2, wherein:
the first well identification feature comprises a first barcode,
the second well identification feature comprises a second barcode, and
the cryogenic storage container further comprises an indicia positioned between the first and second barcodes.

7. The cryogenic storage container of claim 2, wherein the first and second well identification features are configured for human interpretation, computer interpretation, or a combination thereof.

8. The cryogenic storage container of claim 1, wherein the tray further comprises an identifying corner having a different shape than other corners of the tray, the identifying corner configured to indicate an orientation of the tray for identifying the first well in relation to the second well.

9. The cryogenic storage container of claim 1, wherein the tray further comprises an identifying border around the first well, the identifying border configured to indicate an orientation of the tray.

10. The cryogenic storage container of claim 1, further comprising a base having a first pocket and a second pocket sized and positioned to respectively receive the first and second wells therein, wherein the base is configured to transfer heat away from the first and second wells.

11. The cryogenic storage container of claim 1, further comprising a first perforation extending through one or more of the first wall or the first bottom portion, and a second perforation extending through one or more of the second wall or the second bottom portion.

12. The cryogenic storage container of claim 1, further comprising a storage container identification feature on a surface of the tray or the lid, wherein the storage container identification feature is configured to provide information related to the first and second tissue samples contained in the first and second wells.

13. A cryogenic storage container, comprising:
a tray, having
a first well configured to receive a first tissue sample therein;
a first well identification feature associated with the first well;
a second well configured to receive a second tissue sample therein; and
a second well identification feature associated with the second well; and
a lid hingedly associated with the tray and movable between (a) a first position in which the first and second wells are occluded by the lid, and (b) a second position in which the first and second wells are externally accessible,
wherein the first well identification feature is unique from the second well identification feature.

14. The cryogenic storage container of claim 13, wherein:
the first well defines a first opening, and the first well further comprises a first wall projecting from a perimeter of the first opening, and a first bottom portion coupled to the first wall opposite the first opening, and
the second well defines a second opening, and the second well further comprises a second wall projecting from a perimeter of the second opening, and a second bottom portion coupled to the second wall opposite the second opening.

15. The cryogenic storage container of claim 14, further comprising a first perforation extending through one or more of the first wall or the first bottom portion, and a second perforation extending through one or more of the second wall or the second bottom portion.

16. The cryogenic storage container of claim 13, wherein the first and second well identification features are configured to provide information related to one or more of content of the first and second wells, a position of the first and second wells relative to the tray, or a position of the first well relative to the second well.

17. The cryogenic storage container of claim 13, wherein the first and second well identification features comprise one or more of an indicia, a barcode, a matrix code, a cut, a marking, a color code, a letter, a number, or a symbol.

18. The cryogenic storage container of claim 13, wherein:
the first well identification feature comprises a first color at least partially surrounding the perimeter of the first opening,
the second well identification feature comprises a second color at least partially surrounding the perimeter of the second opening, and
the first color is a different hue than the second color.

19. The cryogenic storage container of claim 13, wherein:
the first well identification feature comprises a first barcode,
the second well identification feature comprises a second barcode, and
the cryogenic storage container further comprises an indicia positioned between the first and second barcodes.

20. The cryogenic storage container of claim 13, wherein the first and second well identification features are configured for human interpretation, computer interpretation, or a combination thereof.

21. The cryogenic storage container of claim 13, further comprising a base having a first pocket and a second pocket sized and positioned to respectively receive the first and second wells therein, the base configured to transfer heat away from the first and second wells.

22. The cryogenic storage container of claim 13, further comprising a storage container identification feature on a surface of the tray or the lid, wherein the storage container identification feature is configured to provide information related to the first and second tissue samples contained in the first and second wells.
